(19) United States
(12) Patent Application Publication
(10) Pub. No.: US 2008/0023002 A1
(43) Pub. Date: Jan. 31, 2008

(54) Head safety device with integrated display

(75) Inventors: Thomas Kent Guelzow, Eau Claire, WI (US); Kurt J. Fenske, Gleason, WI (US)

Correspondence Address: MERCHANT & GOULD PC, P.O. BOX 2903, MINNEAPOLIS, MN, 55402-0903, US

(73) Assignee: Rex Systems, Inc.

(21) Appl. No.: 11/881,218

(22) Filed: Jul. 26, 2007

Related U.S. Application Data
(60) Provisional application No. 60/833,339, filed on Jul. 26, 2006.

(51) Int. Cl. A62B 18/00; A42B 3/00; A61B 5/00

(52) U.S. Cl. 128/201.24; 2/5; 600/301

(57) ABSTRACT

A safety device is disclosed. The safety device includes a protective component wearable on a user's head. The safety device further includes a display unit configured to present status information at position in the periphery of the field of view of the user. The safety device includes a communications unit connectable to a wireless communication network and configured to send or receive status information.

In certain embodiments, the safety device is a breathing apparatus or a head protection device. A method of presenting information to a user of the breathing apparatus is also disclosed.

### CROSS REFERENCE TO RELATED APPLICATIONS

The present application claims priority to U.S. Provisional Patent Application Ser. No. 60/833,339, filed Jul. 26, 2006 and entitled “Breathing Apparatus With Integrated Display”, the disclosure of which is hereby incorporated by reference in its entirety.

### TECHNICAL FIELD

The present invention is directed generally to safety devices having sensing and/or communications systems. More particularly, the present invention relates to a head safety device with an integrated display.

### BACKGROUND

Fires, explosions, and other catastrophic events require the action of trained first responders and other emergency responders, including the police department, fire department, and appropriate medical responders. During such events, conditions such as flying/falling debris, smoke, gases, open fires, or other hazardous conditions are likely to be present. These conditions present a hazard to first responders, and can hinder response efforts due to their interference with visual and audio communications between responders.

In many emergency response situations, safety devices, such as head safety devices, helmets, and portable breathing apparatus, are used to protect the emergency responders and others from a variety of hazardous conditions often occurring at an emergency site. Breathing apparatus worn in such situations cover a wearer's entire face with a transparent shield. A portion of such apparatus will generally block some of the periphery of a first responder's visual field. Likewise, helmets can obscure a portion of the field of view of the responder.

Visual indicators incorporated with such protection equipment combine with other conditions at the emergency site to interfere with the first responders' situational awareness. For example, in extreme situations smoke and debris can limit the visual range of a first responder to a few feet in any direction. Use of a breathing apparatus, particularly one including an additional display structure positioned external to the transparent shield, operate in combination with smoke, dust, or other conditions at the emergency site to block a large amount of a first responder's vision. The same is true of helmets, which can obscure lateral vision.

For these and other reasons, improvements are desirable.

### SUMMARY

In accordance with the present disclosure, the above and other problems are solved by the following:

According to a first aspect, a safety device for a user is disclosed. The safety device includes a protective component wearable on a user's head. The safety device further includes a display unit configured to present status information at position in the periphery of the field of view of the user. The safety device includes a communications unit connectable to a wireless communication network and configured to send or receive status information.

According to a second aspect, a breathing apparatus is disclosed. The breathing apparatus includes a protective shield presenting a field of view and a ventilation portion. The breathing apparatus further includes a display unit configured to present information at position in the periphery of the field of view. The breathing apparatus also includes a communications unit connectable to a wireless communication network and configured to send or receive status information.

According to a third aspect, a method of presenting information to a user of a breathing apparatus having a protective shield and a ventilation portion is disclosed. The method includes associating one or more peripheral devices with a display configured to present information at position peripheral to the user's field of view. The method further includes receiving information from the one or more peripheral devices. The method also includes presenting the information using the display.

According to a fourth aspect, a head protection device for an emergency responder is disclosed. The head protection device includes a head protection portion and a display unit mounted on the head protection portion and configured to present information at position in the periphery of a user's field of view. The head protection device includes a communications unit connectable to a wireless communication network and configured to send or receive status information.

### BRIEF DESCRIPTION OF THE DRAWINGS

FIG. 1 is a perspective view of the breathing apparatus according to a possible embodiment of the present disclosure;

FIG. 2 is a view of the breathing apparatus of FIG. 1 in use;

FIG. 3 is a perspective view of a head protection device according to a possible embodiment of the present disclosure;

FIG. 4 is a side perspective view of the device of FIG. 3;

FIG. 5 is a perspective view of a head protection device according to a further possible embodiment of the present disclosure;

FIG. 6 is a side perspective view of the device of FIG. 5;

FIG. 7 is a schematic view of a heads up display within a safety device according to a possible embodiment of the present disclosure;

FIG. 8 is a communication flow diagram of use of the breathing apparatus used at an emergency site, according to a possible embodiment of the present disclosure;

FIG. 9 is a block diagram of a generalized computing system usable to implement aspects of the present disclosure;

FIG. 10 is a logical block diagram of circuitry integrated into a breathing apparatus according to a possible embodiment of the present disclosure;

FIG. 11 is a logical block diagram of the display unit of the breathing apparatus of FIG. 10; and

FIG. 12 is a logical flow diagram of methods and systems for manufacturing and using a breathing apparatus, according to a possible embodiment of the present disclosure.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting, and merely set forth some of the many possible embodiments for the appended claims.

The present disclosure, in general, is related to a safety device incorporating an integrated display. The safety device can be, for example, a helmet or a self contained breathing apparatus connectable to a source of breathable gases, such as air or some other mixture having a sufficient amount of oxygen. The integrated display is a binocular display, and can be, for example, a heads up display appearing to reside a short distance from the wearer of the safety device.

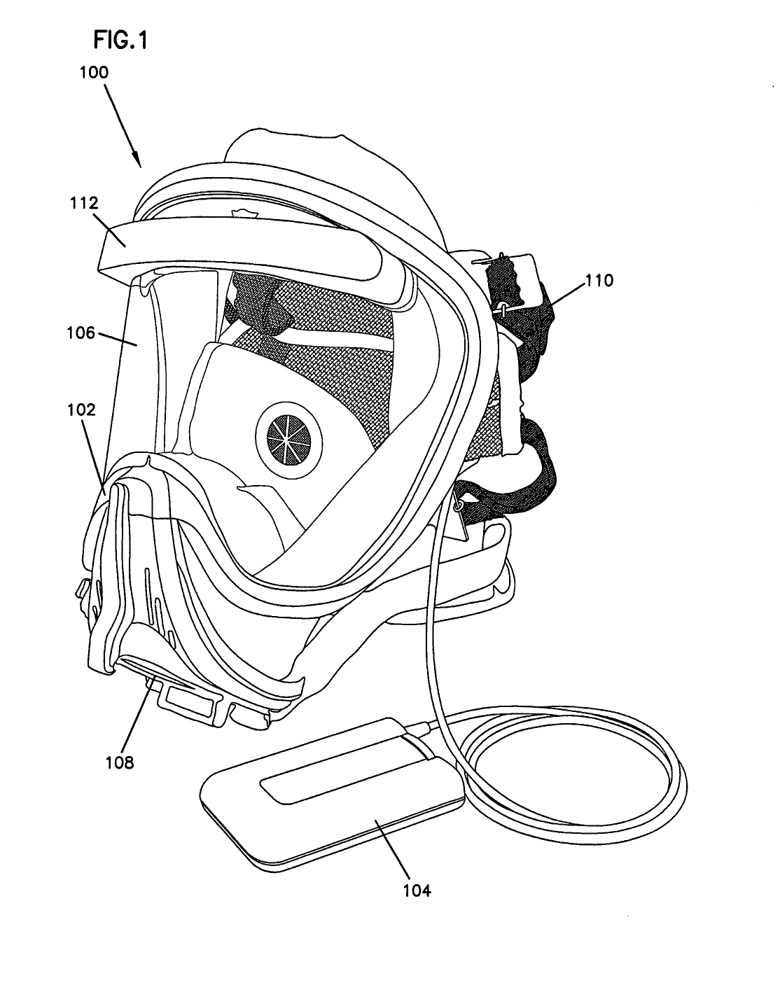

Referring now to FIGS. 1-6, various safety devices are shown in accordance with various embodiments of the present disclosure. In general, the safety devices incorporate one or more heads up displays positioned along a periphery of a user's sight, such that status information can be communicated to the user. Users of the safety devices can include, for example, first responders, military personnel, firefighters, or other emergency response personnel.

Referring now to FIG. 1, a breathing apparatus 100 is shown according to a possible embodiment of the present disclosure. The breathing apparatus 100 is wearable by a first responder during any of a number of emergency or hazardous situations. The breathing apparatus 100 includes a mask portion 102 and a control unit portion 104.

The mask portion 102 is configured for protection of a first responder or other user's face from smoke, gas, debris, or other irritants or hazards. In a possible embodiment, the mask portion is a self contained breathing apparatus, such as are manufactured by MSA, Inc. In the embodiment shown, the mask portion 102 includes a protective shield 106, a ventilation portion 108, head straps 110, and a display unit 112. The protective shield forms an airtight and shatter/puncture resistant transparent guard configured to cover the face of the user. The ventilation portion 108 provides an air inlet/outlet, and can include a filter portion or a connection configured to fit a hose or other conduit to a breathable air reservoir or other oxygen source, and covers the nose and mouth of the user. The head straps 110 hold the protective shield 106 in place over the user's face, and can be adjustable to fit one or more users of the breathing apparatus 100. In other possible embodiments, the mask portion can be another type of mask, such as a respiration hood or other generalized respiration facepieces. The display unit 112 presents a variety of information to the user, and is positioned in the periphery of the field of view available through the protective shield 106. In a possible embodiment, the display unit 112 is a heads up display.

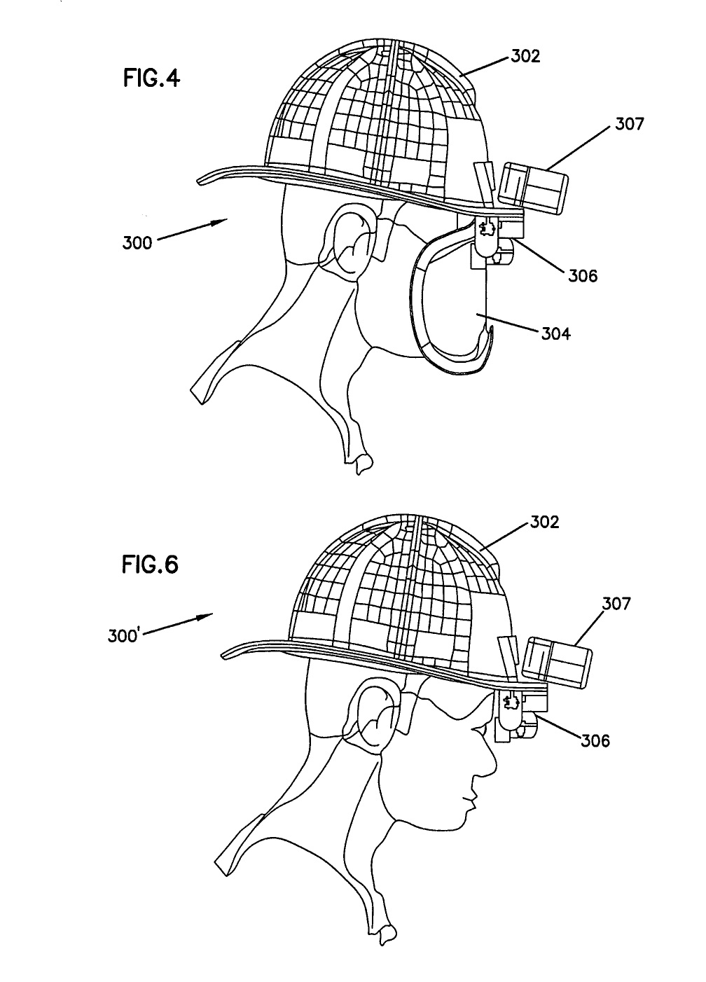

The control unit portion 104 controls the display unit 112 integrated into the mask portion. The control unit portion 104 includes one or more buttons configured to activate or select between a number of display options. In one possible embodiment, the display is a heads up display, such as is described in U.S. Pat. No. 6,879,443, assigned to MicroOptical Corporation, which was filed Sep. 9, 2003, issued Apr. 12, 2005, and entitled Binocular Viewing System, the disclosure of which is incorporated herein in its entirety. In a possible embodiment, the control unit portion 104 contains an interface to or is integrated with a portable computing system (not shown) wearable by the user of the breathing apparatus 100. The portable computing system can control or contain an interface to a mesh network for controlling communications between users of multiple apparatus 100 or standalone computing systems, as described below in conjunction with FIG. 4.

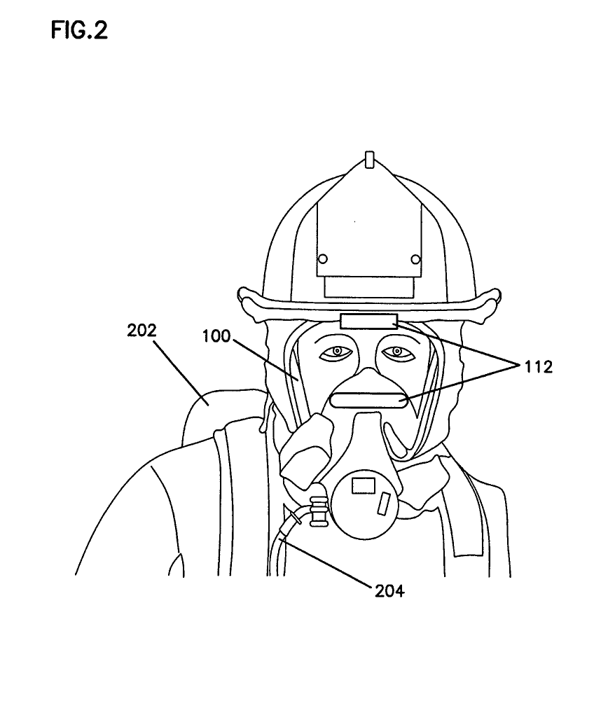

Referring now to FIG. 2, the breathing apparatus 100 of FIG. 1 is shown in use by fire response personnel. The breathing apparatus 100 is wearable in conjunction with additional protective headgear, as necessary. In the embodiment shown, the breathing apparatus 100 is worn by fire response personnel under a protective fire helmet. The breathing apparatus 100 is connected to an oxygen tank 202 worn by the fire response personnel via a hose 204 connected to the ventilation portion 108.

The breathing apparatus includes a display unit 112, depicted as a heads up display, which is mountable within the breathing apparatus 100 either along the ventilation portion 108, the top of the breathing apparatus 100, or both, so as to minimize the visible area blocked by opaque equipment within the apparatus 100. In one embodiment, the display unit 112 generates a heads up display which can include a variety of information, including, for example, the amount of breathable air remaining in the oxygen tank, the current air temperature, the fire response personnel's body temperature, the fire response personnel's heart rate, an infrared representation of the viewable area in front of the mask, battery life of the display, the fire response personnel's location or tracking information, or other options. The display unit 112 presents information to the user such that it appears within the viewable range of the mask and at a distance which can be easily viewed by the fire response personnel. In one possible embodiment, the heads up display information appears to be 3-5 feet from the user.

Optionally, the fire response personnel can also wear sensors and/or communications equipment which can be associated with the breathing apparatus 100 via an interface located either on the mask portion 102 or the control unit portion 104. Each of the sensors and/or communications equipment can be associated with one or more graphical or textual elements presentable to the fire response personnel via the display unit 112. Exemplary sensors associable with the breathing apparatus are described in greater detail below in conjunction with FIG. 6.

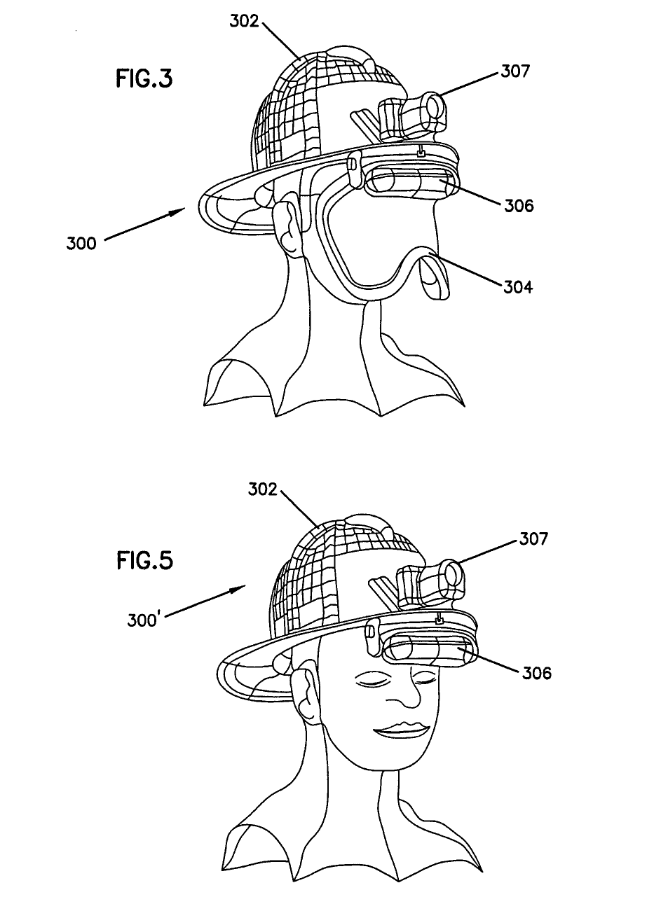

FIGS. 3-6, in general, illustrate various embodiments of a head protection device 300, 300′ with which a heads up display is integrated, according to the present disclosure. Although the protection devices 300, 300′ are shown as a fire helmet, the head protection device can also be any of a variety of other types of helmets, such as a construction helmet, an urban search and rescue head protection device, a SWAT helmet, a mining helmet, or an emergency medical responder's head protection. Additional head protection devices are possible as well.

FIGS. 3-4 show a head protection device 300 according to a possible embodiment of the present disclosure. The head protection device 300 includes a head covering portion 302 and a mask portion 304. The head covering portion 302 protects the user's head, and can take a variety of forms depending upon the chosen application of the device 300. In various embodiments, the head covering portion 302 can be a traditional construction helmet, an urban search and rescue head protection device, a SWAT helmet, a mining helmet, or an emergency medical responder's head protection. The mask portion provides a transparent viewing window that protects the face of a user and allows the user to view his surroundings. The mask portion 302 is, in function, analogous to the protective shield 106 of the breathing apparatus 100 of FIGS. 1-2.

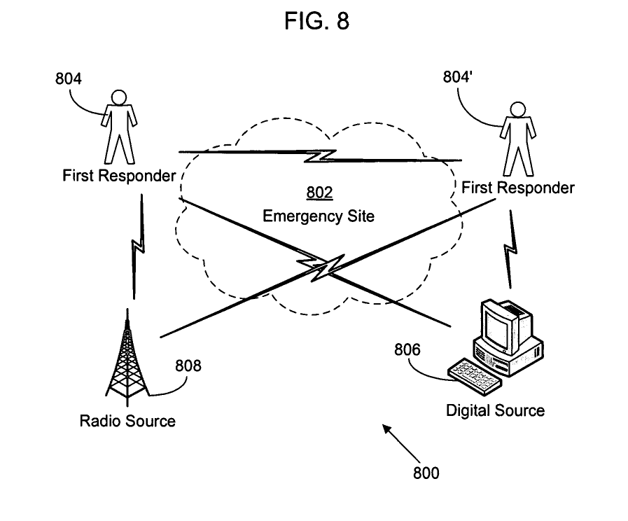

The head protection device 300 further includes a display unit 306. The display unit 306 includes a variety of sensing, communication, and display components, providing the user of the device 300 a variety of information while the device 300 is in use. The display unit 306 can gather a variety of information about the user's surroundings and can display that information to the user in an understandable form. Example information communicated to the user can include: surrounding temperature, body temperature, radiation or gas alarms, communication signal strength, location information (e.g. GPS based), or battery life of the display unit. Additionally, text messages among the device 300, a central server, and other users of the system (as described in FIGS. 8-10, below) may include situational instructions, location and position information of the user or other individuals, directional information, blueprints or maps of relevant buildings, or directions to other individuals. Other information can be displayed by the unit 306 or generated by the device 300 in general as well.

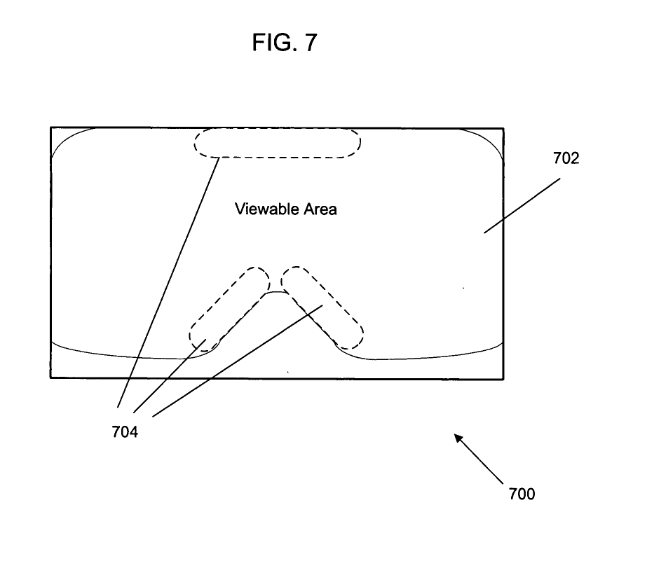

The display unit 306, as shown, is attached to a front portion of the head protection device 300 at the intersection of the head covering portion 302 and the mask portion 304. By locating the display device at this location, the display appears to a wearer of the device 300 at a top periphery of that user's sight. Additional display regions may be integrated into the head protection device as well, such as in the locations described below in conjunction with FIG. 7.

In the embodiment shown, the display unit 306 includes a camera 307, which may be a visual camera, infrared camera, or thermal camera allowing additional sight assistance in instances where vision may be obscured, such as by smoke or darkness. In various embodiments, the display unit 306 or a portion thereof (e.g. the camera or other sensors) is detachably connected to the head protection device 300, allowing modular configuration of components for connection to the device 300. In further embodiments, the display unit 306 and associated components are integrally formed with the head protection device, such as to either the head covering portion 304 or the mask portion.

Additionally, a control unit portion (not shown) may be incorporated into the device 300 or may be interfaced to the device to control the display unit 306. The control unit portion may be, in various embodiments, analogous to the control unit portion 104 of the apparatus 100 of FIG. 1.

Referring now to FIGS. 5-6, a head protection device 300′ is shown according to a further possible embodiment of the present disclosure. The head protection device 300′ generally corresponds to the head protection device 300 of FIGS. 3-4, but does not include the mask portion 304. The head protection device 300′ can be used, for example, with detachable mask portions or legacy breathing apparatus which do not otherwise include heads up displays, or in situations where facial protection is not necessary.

FIG. 7 illustrates a schematic view 700 within a safety device according to a possible embodiment of the present disclosure. The schematic view 700 illustrates possible locations for heads up display components according to various embodiments of the present disclosure. The schematic view 700 illustrates a viewable area 702 including one or more display regions 704. The viewable area 702 is defined by the size and location of the safety device. In the case of the breathing apparatus of claim 1, the viewable area is limited by the protective shield 106, and is limited at a lower portion by the ventilation portion 108. In other situations, the viewable area is limited by facial features or protruding surfaces of a head protection device or other safety device with which the systems of the present disclosure are integrated.

The display regions 704 provide a location at which a heads up display is projected, and are oriented such that the heads up display information is displayed along the bridge or lower central portion of the user's visual field. The displayed information in the display regions 704 is readily available yet does not obstruct the normal vision of the wearer of the safety device, such as a breathing apparatus 100 or head protection device 300 or 300′ of FIGS. 1-6. In one embodiment, one or more of the possible display regions 704 are used. For example, display region 704 at the upper portion of the viewable area 702 can be used in the absence of the display regions along the lower portion of the viewable area 702, such as in the case where the heads up display is connected to a head protection device 300 or 300′ of FIGS. 3-6. Alternately, one or both of the display regions 704 along the ventilation portion 108 can be used alongside or in the absence of the upper display region 704, such as in the case of the portable breathing apparatus 100 of FIGS. 1-2.

In an exemplary embodiment, the display regions 704 present a binocular heads up display to the user of the safety device. The binocular display presents the displayed information to both of the user's eyes, removing a need for special corrective lenses or wearer adjustment. Also, the binocular view maintains a large percentage of natural vision of the surrounding environment.

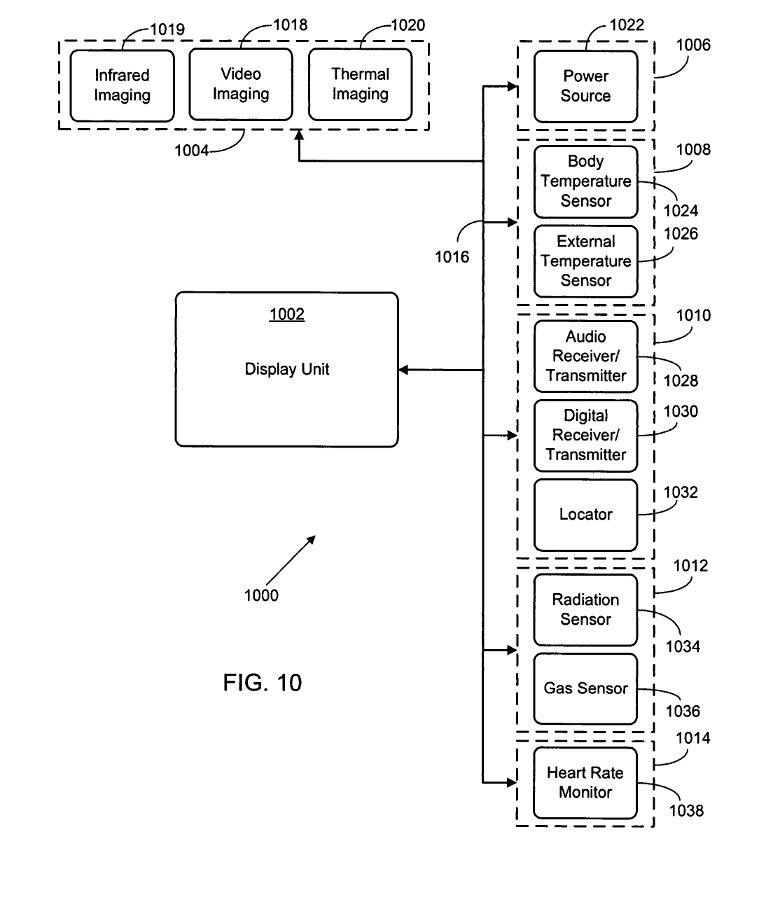

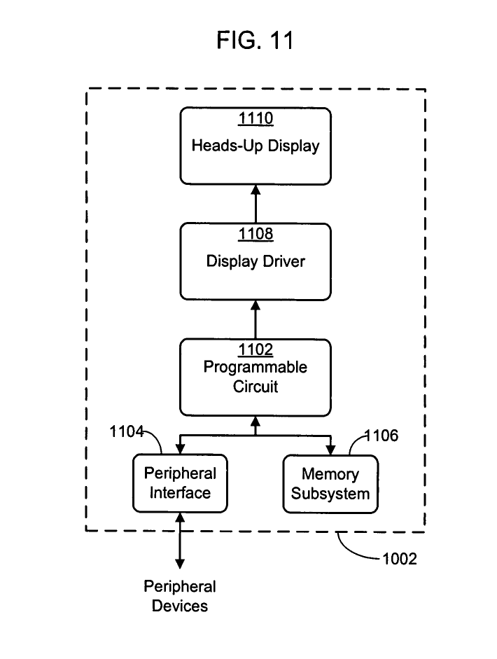

FIG. 8 is a communication flow diagram 800 illustrating use of a safety device at an emergency site 802, according to a possible embodiment of the present disclosure. A plurality of first responders 804, 804′ or other users at the emergency site 802 wear the breathing apparatus 100 of FIGS. 1-2 or head protection device 300 or 300′ of FIGS. 3-6, each of which can include a plurality of sensors and communication devices. The plurality of sensors can detect a number of personal and environmental conditions experienced by the first responder, as described below in conjunction with FIGS. 10-11. The safety devices can each transmit and receive information from one or both of a digital communications source 806 and an audio communications source 808 via the communication devices. In another possible embodiment the digital communications source 806 and/or the audio communications source 808 can be incorporated into a computing system or communication system associated with and recognizable to the safety devices. In a possible embodiment, the digital communications source 806 and/or the audio communications source 808 can be incorporated into a second safety device (of the same or different type) worn by a different first responder 804′. Integration of communications systems into the breathing apparatus provides a convenient way to communicate without requiring that the first responders 404, 404′ remove their head protection equipment or breathing apparatus.

In an exemplary embodiment, the digital communications source 806 or audio communications source 808 includes a mesh radio network configured to transmit messages between first responders at an emergency site 802 and to a central emergency response coordination location (not shown).

In use, the first responders 804, 804′ communicate with each other and with the digital communications source 806 and/or audio communications source 808 about the condition of the emergency at their respective locations. For example, first responder 804 can correspond to first responder 804′ or other communicatively connected individuals about the situation experienced by that individual. The first responder 804 can use an audio or digital communication system for voice or data/text communication, as described below in FIG. 10. The first responder 804′ receiving the message uses a complementary receiver/transmitter to receive and/or reply to the message.

Additionally, the first responders 804, 804′ optionally transmit information from the location of that responder to either the digital communications source 806 or audio communications source 808, for storage or transmission of that data. Other individuals not present at an emergency site, or more generally remote from a user of a device, can access one or both sources 806, 808 to observe transmitted information from a user, such as sensor information, video feeds, or other information. Alternately, one or more of the first responders 804, 804′ can receive information from the sources 806, 808 which is stored there by other first responders or users in general.

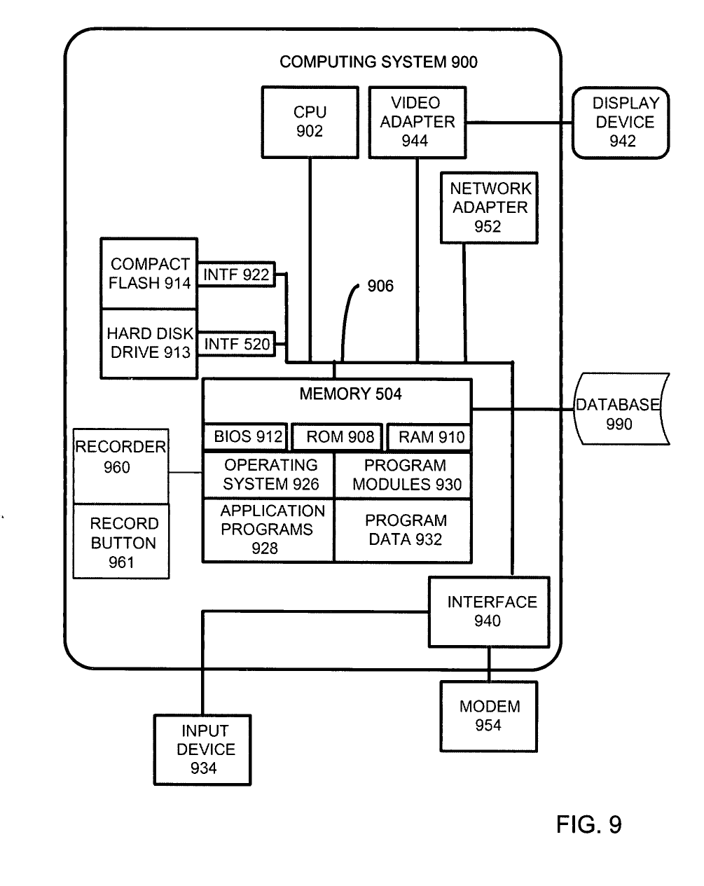

FIG. 9 is a logical block diagram of a generalized computing system usable to implement aspects of the present disclosure. The computing system 900 can be used in communicative connection with the breathing apparatus 100 or head protection device described above, as illustrated in FIG. 8. The computing system architecture includes a general purpose computing device in the form of a computing system 900. The computing system 900 can be used, for example, as the computing system or server 806 of FIG. 8, and can execute program modules included in the administrative software or user software disclosed below.

The computing system 900 including at least one processing system 902. A variety of processing units are available from a variety of manufacturers, for example, Intel or Advanced Micro Devices. The computing system 900 also includes a system memory 904, and a system bus 906 that couples various system components including the system memory 904 to the processing unit 902. The system bus 906 may be any of a number of types of bus structures including a memory bus, or memory controller; a peripheral bus; and a local bus using any of a variety of bus architectures.

The system memory 904 can include read only memory (ROM) 908 and random access memory (RAM) 910. A basic input/output system 912 (BIOS), containing the basic routines that help transfer information between elements within the computing system 900, such as during start up, is typically stored in the ROM 908.

The computing system 900 can also include a secondary storage device 913, such as a hard disk drive, for reading from and writing to a hard disk (not shown), and/or a compact flash card 914.

The hard disk drive 913 and compact flash card 914 are connected to the system bus 906 by a hard disk drive interface 920 and a compact flash card interface 922, respectively. The drives and cards and their associated computer readable media provide nonvolatile storage of computer readable instructions, data structures, program modules and other data for the computing system 900.

Although the exemplary environment described herein employs a hard disk drive 913 and a compact flash card 914, other types of computer-readable media, capable of storing data, can be used in the exemplary system. Examples of these other types of computer-readable mediums include magnetic cassettes, flash memory cards, digital video disks, Bernoulli cartridges, CD ROMS, DVD ROMS, random access memories (RAMs), or read only memories (ROMs).

A number of program modules may be stored on the hard disk 913, compact flash card 914, ROM 908, or RAM 910, including an operating system 926, one or more application programs 928, other program modules 930, and program data 932. A user may enter commands and information into the computing system 900 through an input device 934. Examples of input devices might include a keyboard, mouse, microphone, joystick, game pad, satellite dish, scanner, digital camera, touch screen, and a telephone. These and other input devices are often connected to the processing unit 902 through an interface 940 that is coupled to the system bus 906. These input devices also might be connected by any number of interfaces, such as a parallel port, serial port, game port, or a universal serial bus (USB). Wireless communication between input devices and interfaces 940 is possible as well, and can include infrared, bluetooth, 802.11a/b/g, cellular, or other radio frequency communication systems. A display device 942, such as a monitor or touch screen LCD panel, is also connected to the system bus 906 via an interface, such as a video adapter 944. The display device 942 might be internal or external. In addition to the display device 942, computing systems, in general, typically include other peripheral devices (not shown), such as speakers, printers, and palm devices.

When used in a LAN networking environment, the computing system 900 is connected to the local network through a network interface or adapter 952. When used in a WAN networking environment, such as the Internet, the computing system 900 typically includes a modem 954 or other communications type, such as a direct connection, for establishing communications over the wide area network. The modem 954, which can be internal or external, is connected to the system bus 506 via the interface 940. In a networked environment, program modules depicted relative to the computing system 900, or portions thereof, may be stored in a remote memory storage device. It will be appreciated that the network connections shown are exemplary and other methods of establishing a communications link between the computing systems may be used.

The computing system 900 might also include a recorder 960 connected to the memory 904. The recorder 960 includes a microphone for receiving sound input and is in communication with the memory 904 for buffering and storing the sound input. The recorder 960 also can include a record button 961 for activating the microphone and communicating the sound input to the memory 904.

Optionally, the computing system 900 further includes a database 990 configured to store one or more types of data, and can be configured to gather information from various sources connected to the system.

A computing device, such as computing system 900, typically includes at least some form of computer-readable media. Computer readable media can be any available media that can be accessed by the computing system 900. By way of example, and not limitation, computer-readable media might comprise computer storage media and communication media.

Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium that can be used to store the desired information and that can be accessed by the computing system 500.

Communication media typically embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media. Computer-readable media may also be referred to as computer program product.

FIG. 10 illustrates a logical block diagram of circuitry for a safety device 600 incorporated into or alongside a safety device such as a breathing apparatus or head protection device (as illustrated in FIGS. 1-6, above) according to a possible embodiment of the present disclosure. The safety device 1000 includes a display unit 1002 configured to generate a heads up display, such as the one shown above in FIG. 7. The display unit is described in greater detail below, in conjunction with FIG. 11. The display unit 1002 is associated with a plurality of peripheral devices, including an imaging unit 1004, a power unit 1006, a temperature sensor unit 1008, a communications unit 1010, a hazard sensor unit 1012, and a vital signs unit 1014. The plurality of peripheral devices 1004-1014 are mountable on the breathing apparatus 100 of FIGS. 1-2 or head protection device 300, 300′ of FIGS. 3-6, or elsewhere among the portable equipment carried by emergency response personnel. The peripheral devices 1004-1014 can be communicatively connected via a communications bus 1016 with either a mask portion or a communications unit portion of the safety device 1000.

The imaging unit 1004 detects and generates a digital representation of the visual state in front of a sensor. In one embodiment, the imaging unit includes a video imaging subunit 1018 and/or a thermal imaging subunit 1020 mounted on the safety device, facing outwardly toward the field of view of a user. The video imaging subunit 1018 generates a streaming video of scene visible to a user of the breathing apparatus 1000. The thermal imaging subunit 1020 generates a thermal image of the same scene. The user of the safety device 1000 selectably views or transmits the images produced by either subunit 1018, 1020. Example information communicated to the user by the imaging unit can include: surrounding temperature, body temperature, radiation or gas alarms, communication signal strength, location information (e.g. GPS based), or battery life of the display unit. Additionally, and as previously mentioned, text messages among the device, a central server, and other users of the system (as described in FIGS. 8-10) may include situational instructions, location and position information of the user or other individuals, directional information, blueprints or maps of relevant buildings, or directions to other individuals.

The power unit 1006 includes a power source 1022, which provides power to the safety device 1000. In various embodiments, the power source can be a battery or other portable power source, or can be a connection to an external power source, such as an external battery pack. The power unit 1006 also optionally includes circuitry configured to detect the remaining capacity of the power source, and to communicate the capacity of the power source to the display unit to be presented to the user.

The temperature sensor unit 1008 detects temperature at one or more locations, and communications the sensed temperature to the display unit to be presented to the user. In the exemplary embodiment shown, the temperature sensor unit includes a body temperature sensor 1024 and an external temperature sensor 1026. The body temperature sensor 1024 resides proximate to the user's body, and measures and transmits the user's current body temperature for display by the display unit 1002. The external temperature sensor 1026 resides exposed to the external temperature and in a position unaffected by radiant heat from a user. The external temperature sensor 1026 measures and transmits the environmental temperature to the display unit 1002. In a possible embodiment, the external temperature sensor 1026 can generate an alert to warn the user of the safety device 1000 of exposure to dangerously high or low temperatures.

The communications unit 1010 provides audio and/or digital communications integrated into the safety device 1000, thereby avoiding the requirement that the apparatus be removed during communication. The communications unit can include a variety of communication devices capable of communicating using a variety of protocols, and in the embodiment shown includes an audio receiver/transmitter 1028, a digital receiver/transmitter 1030, and a locator 1032. The audio receiver/transmitter 1028 can be any analog wireless radio communication system, such as a 900 MHz or 2.4 GHz radio band signal. The digital receiver/transmitter 1030 can be any type of synchronous or asynchronous digital transmission device, such as a cellular communication unit, an 802/11 a/b/g unit, or other digital communications unit. The locator unit 1032 can be, for example, a global positioning system (GPS) or non-GPS unit (such as a time of arrival system or other location tracking system) configured to compute and display or transmit the location of the safety device 1000. The communications unit 1010 transmits to the display unit 1002 a communications signal strength for each of the communications devices included in the communications unit.

By using the communications unit 1010, a user can send and receive voice or text messages to other users of analogous devices, either directly from device to device, or relayed by a server such as the one described in FIGS. 8-9. This can be accomplished by use of a text or voice recognition input device, such as a receiver or keyboard (not shown). Additionally, the communications unit 1010 can transmit information captured by the various sensors and video cameras integrated into the safety device 1000 to a central server or another individual, such as within the network of FIG. 8. Other communication capabilities may be provided by the communications unit as well.

The hazard sensor unit 1012 detects potentially hazardous conditions in the vicinity of the safety device 1000. In the embodiment shown, the hazard sensor unit 1012 includes a radiation sensor 1034 and a gas sensor 1036. The radiation sensor 1034 detects the level of radiation currently present in the area of the device 1000. The gas sensor 1036 is configurable to detect one or more hazardous gases, such as flammable or otherwise lethal gases. Other sensors can be incorporated into the hazard sensor unit 1012 as well.

The vital signs unit 1014 monitors the user of the safety device 1000, and can include a heart rate monitor 1038 configured to observe and communicate the user's heart rate to the display unit 1002. Other monitors, such as a breathing monitor incorporated into a ventilation portion of a mask portion of a breathing apparatus 100 (seen in FIG. 1) can be included as well, and depend upon the specific configuration of the safety device 1000.

In various embodiments, the units 1006-1014 are configurable to reside either in the mask portion or in a control unit portion of the apparatus 100 (seen, for example, in FIGS. 1-2). In further embodiments, the units 1006-1014 are configured for attachment to a front portion of a head protection device 300, 300′ as shown in FIGS. 3-4 and 5-6. In one possible embodiment, the units 1006-1014 are integrated into a wearable computing system incorporated into or interfaced with a control unit, such as the control unit portion 104 of FIG. 1. The imaging unit 1004 resides within the mask portion and is oriented toward the area visible to the user of the safety device 600.

FIG. 11 illustrates a logical block diagram of the display unit 1002 of the safety device of FIG. 10. In general the display unit is a binocular display unit, meaning that the display unit is configured to produce images which are displayed to both of a user's eyes and cause the displayed images to appear to be at a specified distance away from the user due to optical triangulation and image positioning. The display unit 1002 includes a programmable circuit 1102 interfaced to a memory subsystem 1104 and a peripheral interface 1106. The programmable circuit 1102 is further interfaced to a display driver 1108, which in turn generates a sufficient signal to be displayed on a heads up display 1110.

The programmable circuit 1102 can be any embedded processor, programmable gate array, or other programmable logic device. The programmable circuit 1102 is configured to execute instructions stored in the memory subsystem 1104, which can be any of a number of types or combinations of volatile and nonvolatile memory. The instructions define the operation of the display unit 1002, allowing a user to change the operation of the display unit by altering the software stored in the memory subsystem 1104. The display driver 1108 receives display information from the programmable circuit 1102, and generates a video signal configured for use on the heads up display 1104.

In a possible embodiment, the display driver includes two display drivers configured to create a pair of images which a user visually triangulates so as to focus on a point a few feet away. Generation of this optical effect is described in greater detail in U.S. Pat. No. 6,879,443, assigned to MicroOptical Corporation, which was filed Sep. 9, 2003, issued Apr. 12, 2005, and entitled Binocular Viewing System, the disclosure of which was incorporated by reference above.

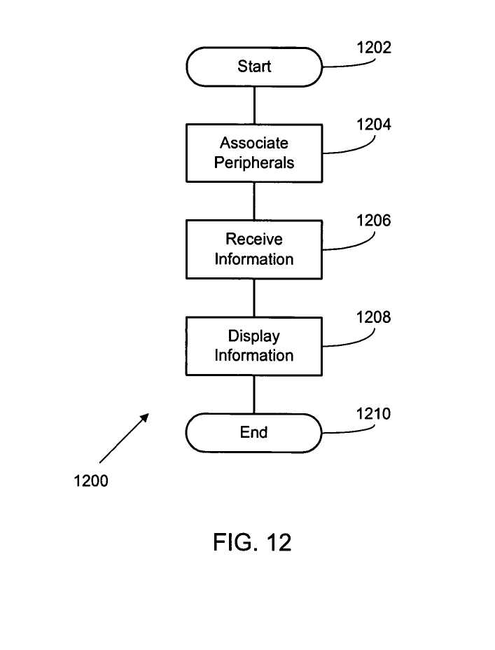

FIG. 12 is a logical flow diagram of methods and systems for manufacturing and using a breathing apparatus, according to a possible embodiment of the present disclosure. The system 1200 is instantiated by a start operation 1202. Operational flow proceeds to an associate module 1204. The associate module 1204 associates one or more peripheral devices, such as sensors, telecommunications equipment, air tank pressure gauges, or other devices with a safety device such as a breathing apparatus or head protection device, such as those illustrated in FIGS. 1-10. In an exemplary embodiment, the associate module 1204 includes associating one or more of the peripheral functional units shown in FIG. 9 with a safety device, such as a breathing apparatus 100 shown in FIGS. 1-2 or a head protection device 300, 300′ shown in FIGS. 3-6.

Operational flow proceeds to a receive module 1206. The receive module 1206 receives information from one or more functional units interfaced with a safety device. The information can include air tank capacity information (in the instance that the safety device is a breathing device), data or audio communication signals, temperature readings, or other information. In an exemplary embodiment, the receive module 1206 receives information from one or more of the functional units shown in FIG. 9.

Operational flow proceeds to a display module 1208. The display module 1208 generates a display to be presented to a user of a breathing apparatus. The display module 1208 generates the display from within the breathing apparatus, and presents to the user of the breathing apparatus the information received via the receive module 1206. In one embodiment, the display is a heads up display, such as is described above in conjunction with FIG. 10. Operational flow terminates at an end operation 1210.

The various embodiments described above are provided by way of illustration only and should not be constructed to limit the claims attached hereto. Those skilled in the art will readily recognize various modifications and changes that may be made without following the example embodiments and applications illustrated and described herein, and without departing from the true spirit and scope of the following claims.

1. A safety device for a user, the safety device comprising:
a protective component wearable on a user's head;
a display unit configured to present status information at position in the periphery of a field of view of the user; and
a communications unit connectable to a wireless communication network and configured to send or receive status information.

2. The safety device of claim 1, wherein the protective component is a transparent mask.

3. The safety device of claim 1, wherein the status information includes emergency response information.

4. The safety device of claim 1, wherein the protective component is a helmet.

5. The safety device of claim 1, wherein the display unit includes a plurality of sensors configured to detect environmental conditions.

6. The safety device of claim 1, wherein the display device includes one or more video capture devices.

7. The safety device of claim 1, further comprising a communication unit configured to communicatively connect to a wireless communication network.

8. The safety device of claim 7, wherein the communication unit communicates data detected at the location of the safety device to a computing system communicatively connected to the wireless communication network.

9. A breathing apparatus comprising:
a protective shield presenting a field of view;
a ventilation portion;
a display unit configured to present information at position in the periphery of the field of view; and
a communications unit connectable to a wireless communication network and configured to send or receive status information.

10. The breathing apparatus of claim 9, wherein the display unit is further configured to be located between a user's face and the protective shield.

11. The breathing apparatus of claim 9, wherein the display unit is a heads up display.

12. The breathing apparatus of claim 11, wherein the heads up display is a binocular heads up display.

13. The breathing apparatus of claim 9, further comprising a control unit interfaced with the display unit and configured to control the display unit.

14. The breathing apparatus of claim 9, further comprising one or more peripheral devices interfaced to the breathing apparatus.

15. The breathing apparatus of claim 14, wherein the one or more peripheral devices are selected from the group consisting of:
an imaging unit;
a power unit;
a temperature sensor unit;
a hazard sensor unit; and
a vital signs unit.

16. The breathing apparatus of claim 9, wherein the communication unit communicates data detected at the location of the breathing apparatus to a computing system communicatively connected to the wireless communication network.

17. A method of presenting information to a user of a breathing apparatus having a protective shield and a ventilation portion, the method comprising:
associating one or more peripheral devices with a display configured to present information at position peripheral to the user's field of view;
receiving information from the one or more peripheral devices;
presenting the information using the display.

18. The method of claim 17, wherein the display is located between the protective shield and a user's face.

19. The method of claim 17, wherein presenting the information on the display comprises presenting information on a heads up display.

20. The method of claim 19, wherein the heads up display is a binocular heads up display.

21. The method of claim 17, further comprising communicating the information on a wireless communication network.

22. The method of claim 17, wherein the one or more peripheral devices are selected from the group consisting of:
an imaging unit;
a power unit;
a temperature sensor unit;
a hazard sensor unit; and
a vital signs unit.

23. A head protection device for an emergency responder comprising:
a head protection portion;
a display unit mounted on the head protection portion and configured to present information at position in the periphery of a user's field of view; and
a communications unit connectable to a wireless communication network and configured to send or receive status information.

24. The head protection device of claim 23, wherein the head protection portion comprises a fire helmet.

25. The head protection device of claim 23, further comprising one or more peripheral devices attached to the device.

26. The head protection device of claim 25, wherein the one or more peripheral devices are selected from the group consisting of:
an imaging unit;
a power unit;
a temperature sensor unit;
a hazard sensor unit; and
a vital signs unit.

27. The head protection device of claim 23, wherein the head protection device forms a communication node in an emergency response communication network at an emergency site.

28. The head protection device of claim 23, wherein the communication unit communicates data detected at the location of the device to a computing system communicatively connected to the wireless communication network.

29. The head protection device of claim 23, wherein the display unit includes a binocular heads up display.
